(19) United States
(12) Patent Application Publication
(10) Pub. No.: US 2009/0227994 A1
(43) Pub. Date: Sep. 10, 2009

(54) DEVICE AND METHOD FOR THE DELIVERY AND/OR ELIMINATION OF COMPOUNDS IN TISSUE

(75) Inventors: Warren S. Grundfest, Los Angeles, CA (US); Yaser M. Abdulraheem, Kuwait City (KW); Sze-Chun Chan, Los Angeles, CA (US); Margaret Chiang, Los Angeles, CA (US); Mario K. Furtado, Los Angeles, CA (US); Kevin Geary, Union City, CA (US); Chih-Kang Lin, Los Angeles, CA (US)

Correspondence Address: Vista IP Law Group LLP, 2040 MAIN STREET, 9TH FLOOR, IRVINE, CA, 92614, US

(73) Assignee: THE REGENTS OF THE UNIVERSITY OF CALIFORNIA, Oakland, CA (US)

(21) Appl. No.: 11/573,422

(22) PCT Filed: Aug. 9, 2005
(86) PCT No.: PCT/US05/28210

§ 371 Date: Feb. 8, 2007

Related U.S. Application Data
(60) Provisional application No. 60/600,150, filed on Aug. 10, 2004.

(51) Int. Cl. A61B 18/20; A61B 19/00

(52) U.S. Cl. 606/9; 606/186

(57) ABSTRACT

A device for removing compounds in tissue such as, for example, tattoo pigment compounds in skin tissue includes a detector for detecting the peak optical absorption of the compound, a laser source, wherein the wavelength is tuned or selected based on the peak optical absorption of the compound in the skin. The device further includes a delivery member for delivering radiation from the laser source to the tissue. Compounds such as tattoo pigment compounds are removed by detecting the peak optical absorption of the tattoo pigments or photofragments thereof in tissue with the detector. The wavelength of the laser source is adjusted based on the peak optical absorption of the compound in the tissue, and delivers radiation at the adjusted (or non-adjusted) wavelength from the laser source to the compound in the tissue with the delivery member.

### REFERENCE TO RELATED APPLICATIONS

This Application claims priority to U.S. Provisional Patent Application No. 60/600,150 filed on Aug. 10, 2004. U.S. Provisional Patent Application No. 60/600,150 is incorporated by reference as if set forth fully herein.

### FIELD OF THE INVENTION

The field of the invention generally relates to methods and devices used for the delivery and/or elimination of compounds in tissue. For example, the invention relates to laser-based devices used in the administration and/or removal of certain organic pigment compounds in skin. For instance, the methods and devices may be used in the administration and removal tattoos which may include, for example, cosmetic and/or clinical tattoos. The invention further relates to methods and devices used in the delivery and/or elimination of pharmaceutical compounds or pharmaceutical precursor compounds located in tissue.

### BACKGROUND OF THE INVENTION

The interest in the art of tattooing has been increasing steadily over the past decade. In the United States alone, it is estimated that about 5-10% of the population has some sort of tattoo, either cosmetic or clinical in nature. This increase in demand for tattoos has lead to a commensurate increase in the demand for tattoo removal procedures. Current removal techniques are far from optimized, however, and suffer from the inherent limitation of not knowing the chemical and optical properties of the pigments used in the tattoo ink. This often results in several adverse consequences such as, for example, tissue damage, drastic tattoo darkening, and even incomplete pigment removal (even after repeated treatments).

Currently, Q-Switching (i.e., pulsed) laser tattoo removal is accepted as the primary method for the removal of unwanted tattoo pigments contained in the skin. Current Q-Switching laser systems, however, suffer from a number of limitations. First, current laser systems generally utilize between one and three frequencies to target all pigment colors in the tattoo. Through visual inspection, it can be seen that this approach gives a gradient of results, removing certain pigment colors better than others. This observation suggests that the different pigments respond differently to specific wavelengths and that the current “single-frequency-fits-all” approach may not be the most effective solution.

Second, current laser-based devices and methods used for tattoo removal are unable to identify the targeted pigments. Since different pigments have different optical properties, pigment identification is a crucial step to effectively remove the pigment from the skin. This task is currently extremely difficult because there are no standards in tattoo pigment composition. The U.S. Food and Drug Administration (FDA), for example, does not regulate the use of tattoo pigments nor does the FDA regulate the actual practice of tattooing. As a result, a wide variety of chemical compounds are used for tattoo pigments, some of which are toxic and harmful to the human body. This poses a challenge for laser tattoo removal specialists to safely and successfully remove tattoo pigments.

Finally, the physical mechanisms of laser-tissue and laser-pigment interactions are not well understood and, consequently, are not fully optimized in current removal devices and methods. For example, there are limits to the depth of laser light penetration into tissue. In addition, there are limits on the amount of energy required to remove tattoo pigments without damaging neighboring tissue. These physical restraints on laser systems limit the effectiveness of the system in removing tattoo pigment compounds. Also, because the administration of tattoos is not heavily regulated, the depth in which the pigment particles are implanted underneath the skin surface varies to a large extent (typically ranging from about 0.3 mm to about 1.5 mm). This presents a challenge for tattoo removal because the laser light may not be able to reach the pigment(s) at the desired depth with the optimum wavelength and energy.

There thus is a need for safe and completely removable tattoo pigment compounds. Such a method and/or device for application/removal would have numerous cosmetic and clinical applications. If a safe and removable tattoo were available, cosmetic tattoos would become even more popular because recipients would confidently know that the tattoo they are receiving is readily removable and not permanent. Moreover, a safe and completely removable tattoo has clinical applications. For example, tattoo markers may be used during surgical procedures to mark incision regions and for long-term post-surgical follow-ups.

Apart from tattoos, there is also a need for a safe and effective method for the delivery and/or removal of pharmaceutical compounds to a patient or subject. In some cases, it is desirable to deliver a pharmaceutical compound to a localized region (e.g., cancerous tissue). In many instances, however, it is undesirable (or even impossible) to locally delivery such compounds, for example, via subcutaneous injection. In this case, there is a need for a modality of activating a pharmaceutical compound locally. In still other situations, there may be a need to rapidly eliminate or remove a locally administered pharmaceutical compound. For most pharmaceutical compounds, the elimination of the compound from the body takes place over a relatively long period of time. However, for many compounds that are toxic in nature (e.g., chemotherapeutic agents), there is a desire to reduce the amount of exposure of such compounds to healthy tissues. There thus is a need for a method of rapidly eliminating a pharmaceutical compound from tissue.

### SUMMARY OF THE INVENTION

In one aspect of the invention, a device for removing a compound in tissue such as skin tissue includes a detector for detecting at least one optical property of the compound in the tissue, a laser source, wherein the wavelength of the laser source is based on the at least one optical property of the compound in the tissue, and a delivery member for delivering radiation from the laser source to the compound in the tissue. The at least one optical property may include peak optical absorption information.

In another aspect of the invention, a device for removing tattoo pigment compounds in tissue such as skin includes a detector for detecting the peak optical absorption of one or more of the tattoo pigment compounds in the tissue, a tunable laser source, wherein the wavelength is tuned based on the peak optical absorption of the tattoo pigment compound(s) in the tissue, and a delivery member for delivering radiation from the tunable laser source to the tattoo pigment compounds in the tissue.

In another preferred aspect of the invention, a method of administering a tattoo includes the steps of inserting a pigment into the dermis layer of skin at a known depth level, wherein the pigment is selected from the group consisting of Chicago Sky Blue 6B, Methyl Red, Phenolphthalein, Janus Green B, Crystal Violet, Cresyl Violet Perchlorate, Chrysophenine, and Fast Black K Salt (Azoic Diazo No. 38).

In still another aspect of the invention, a method of removing a tattoo includes the steps of: providing a detector, providing a tunable laser source, providing a delivery member for delivering radiation from the tunable laser source to the tattoo pigment in the skin, detecting the peak optical absorption of the tattoo pigment in the skin with the detector, adjusting the wavelength of the tunable laser source based on the depth and peak optical absorption of the tattoo pigment in the skin, and delivering radiation at an adjusted wavelength from the tunable laser source to the tattoo pigment in the skin with the delivery member.

In still another aspect of the invention, the above-identified method further includes the steps of: detecting the peak optical absorption of photofragments of the tattoo pigment in the skin with the detector, adjusting the wavelength of the tunable laser source based on the peak optical absorption of the photofragments of the tattoo pigment in the skin, and delivering radiation at an adjusted wavelength from the tunable laser source to the photofragments of the tattoo pigment in the skin with the delivery member.

In another aspect of the invention, a system is provided for the delivery and/or removal of one or more pharmaceutical compounds and/or pharmaceutical precursor compounds. In one aspect of the invention, a pharmaceutical compound is administered to a subject. For example, the compound may be locally deposited within tissue. A laser source is used to illuminate the region of skin containing the pharmaceutical compound. The laser radiation interacts with and breaks down the pharmaceutical compound, thereby removing the pharmaceutical compound from the tissue.

In another aspect of the invention, one or more pharmaceutical precursor compounds are administered to a subject. For example, the pharmaceutical precursor compounds may be deposited locally within skin tissue. A laser source is used to illuminate the region of skin containing the one or more pharmaceutical precursor compounds. The laser radiation interacts and transforms the pharmaceutical precursor compound into a compound (or multiple compounds) having therapeutic properties. In this regard, radiation is used to initiate or otherwise trigger or modulate the release of a therapeutic pharmaceutical compound located with tissue. These compounds may have localized or systemic therapeutic effects.

It is an object of the invention to provide an integrated tattoo removal system that uses real-time or near real-time detection techniques to optimally tune or select a wavelength from a laser source. It is a further object of the invention to provide a method for administering and removing tattoo pigment compounds from skin. Further objects of the invention are described in more detail below.

### BRIEF DESCRIPTION OF THE DRAWINGS

FIG. 1 illustrates a device used to remove compounds such as tattoo pigment compounds from tissue such as skin according to one preferred embodiment of the invention.

FIG. 2 illustrates a spectroscopic optical coherence tomography (OCT) detection system.

FIG. 3(a) illustrates a dual wavelength compound fragmentation system using a Nd:YAG laser (532 nm) and a ruby laser (694 nm).

FIG. 3(b) illustrates a tunable compound fragmentation system using a tunable ruby OPO laser system.

FIG. 3(c) illustrates a tunable compound fragmentation system using a tunable Nd:YAG OPO laser system.

FIG. 3(d) illustrates a tunable tattoo fragmentation system using a tunable Ti:Sapphire OPO laser system. (CWML: continuous wave modelock. ML: Modelocker.)

FIG. 4(a) illustrates a selected region of tissue containing a pharmaceutical precursor compound disposed therein.

FIG. 4(b) illustrates the selected region of tissue shown in FIG. 4(a) being irradiated with laser radiation so as to transform at least some of the pharmaceutical precursor compounds into a therapeutic pharmaceutical compound.

FIG. 4(c) illustrates the selected region of tissue shown in FIGS. 4(a) and 4(b) after complete transformation of the pharmaceutical precursor compounds into a therapeutic pharmaceutical compound.

### DETAILED DESCRIPTION OF THE PREFERRED EMBODIMENTS

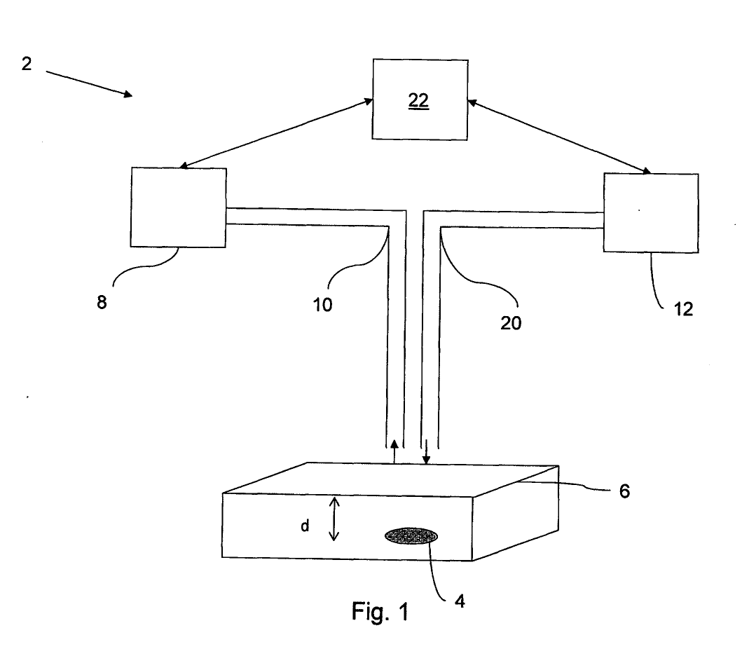

FIG. 1 schematically illustrates a device 2 used to remove or otherwise degrade a compound 4 or plurality of different compounds 4 (or photofragments of a compound(s) 4) contained within tissue 6. In one embodiment of the invention, the tissue 6 includes the dermis layer of skin. It should be understood, however, that the invention may be applied to a variety of tissue 6 types and is not limited to skin. The compound 4 is preferably an organic-based compound and, in one aspect of the invention, may include a pharmaceutical compound or a pharmaceutical precursor compound (discussed in more detail below). The compound 4 may also include a pigment compound such as those used in tattoos.

Referring back to FIG. 1, a representative tattoo pigment compound 4 is contained within tissue 6 (e.g., dermis layer of skin). In a preferred aspect of the invention, the tattoo pigment compound 4 is an organic-based pigment. Even more preferably, the tattoo pigment 4 is one of the following organic-based pigments: Chicago Sky Blue 6B (C₃₄H₂₄N₆Na₄O₁₆S₄), Methyl Red (C₁₅H₁₅N₃O₂), Phenolphthalein (C₂₀H₁₄O₄), Janus Green B (C₃₀H₃₁N₆Cl), Crystal Violet (C₂₅H₃₀ClN₃), Cresyl Violet Perchlorate (C₁₆H₁₂ClN₃O₅), Chrysophenine (C₃₀H₂₆N₄Na₂O₈S₂), and Fast Black K Salt (Azoic Diazo No. 38) (C₁₄H₁₂N₅O₄ 0.5[Cl₄Zn] or C₁₄H₁₄N₅O₄.CI). These compounds represent the following colors, respectively: red, blue, green, yellow, violet, and black. Of course, other organic-based or even non-organic based tattoo pigment compounds 4 may also be used. Generally, pigment compounds 4 that have absorption spectra that can be matched or tuned by electromagnetic radiation from a source such as a laser may be employed. Pigment compounds 4 used in accordance with this invention may include pigment compounds 4 having one or more of the following properties: (1) color permanence and stability in skin (with respect to permanent tattoos), (2) a high degree of bio-compatibility, (3) an absorption spectrum with a strong or relatively strong peak around one of the main laser emission lines (or a tunable wavelength) and an absorption peak far from the UV-melanin absorption wavelength, (4) are completely or nearly completely removed by laser treatment, and (5) have photofragments (pigment compound degradation products) with low levels of toxicity.

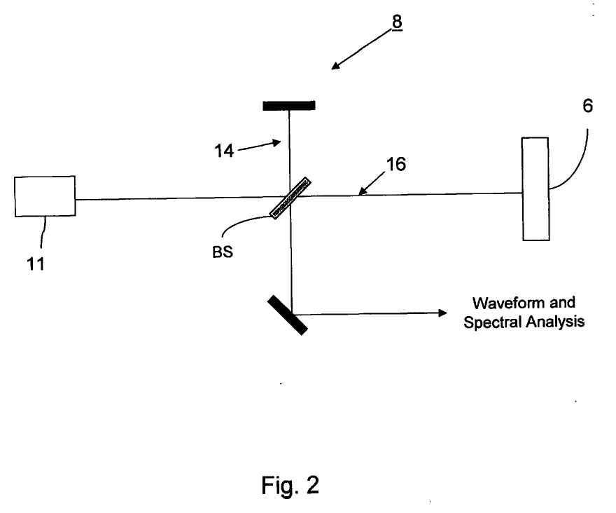

Still referring to FIG. 1, the device 2 includes a detector 8 for detecting the depth and/or peak optical absorption of the compound 4 within the tissue 6. In a one aspect of the invention, the detector 8 includes a detection path 10 where reflected radiation is collected and passed from the surface of the tissue 6 to the actual detector 8. The detection path 10 may include, for example, one or more optical pathways such as an optical fiber or bundle of multiple fibers (e.g., multimode fiber). One skilled in the art will appreciate that the particular detector path 10 used may include any of those commonly used to transport laser radiation from one location to another. In one aspect of the invention, the detector 8 is a spectral optical coherence tomography (OCT) system as is shown in FIG. 2. It should be understood, however, that other detectors 8 capable of detecting the depth and/or peak optical absorption information of a compound 4 within tissue 6 may be used. For example, by way of illustration and not limitation, the detector 8 may include a microscopic-based detector such as a confocal microscope-based detector.

As seen in FIG. 2, the spectral optical coherence tomography (OCT) system includes laser source 11 such as, for example, a low power Ti:Sapphire laser which is split into a reference arm 14 and a sample arm 16 using a beam splitter BS. Other laser sources 11 that may be used in the OCT system may include low coherent or incoherent sources, LED-based sources, or other supercontinuum-type sources. A moveable mirror 15 or the like (such as an optical modulator) may be used to introduce delay in the reference arm 14 consistent with OCT systems. Of course, delay may be introduced in the reference arm 14 using any other devices and/or methods. Reflected radiation from the tissue sample 6 via the sample arm 16 interferes with the reference arm 14 and is subject to waveform and spectral analysis. The methods disclosed in, for example, R. Leitgeb et al., “Spectral measurement of absorption by spectroscopic frequency-domain optical coherence tomography,” Optics Letters, June 2000, Vol. 25(11), pp. 820-822 may be employed to determine the depth (d) of the compounds 4 (or photofragments thereof) as well as the absorption peak(s) of the compounds 4. The R. Leitgeb et al. article is incorporated by reference as if set forth fully herein.

As explained above, the detector 8 is used to determine at least one parameter for a particular compound 4 (e.g., tattoo pigment compound 4). This may include the compound's depth (d) (as seen in FIG. 1) and/or its peak optical absorption. In another embodiment, the detector 8 may be used to determine fluorescence peak information. For example, the compound 4 may fluoresce in response to being irradiated with a particular wavelength of radiation. This information is then utilized, as explained in more detail below, to tune or select a wavelength in a laser source 12 to an optimum or substantially optimum wavelength based on the measured peak optical absorption. It should be understood that laser source 12 does not necessarily have to be tuned to the absolute peak optical absorption of the compound 4. Rather, the laser source 12 may be tuned to be at or near the peak optical absorption of the compound 4. Moreover, it should be understood that compounds 4 may have a plurality of local peak optical absorptions. In this case, the laser source 12 may be tuned to be at or near one of the local peak optical absorptions. This may or may not be a global maximum peak optical absorption.

In one embodiment, the depth (d) is used to aim the radiation from the laser source 12 at the compound 4 at the optimum location within the tissue 6 for photofragmentation. The depth of penetration from the laser source 12 may be accomplished by adjusting the focal point of the laser, for example, by adjusting the longitudinal position of a focusing lens.

In another embodiment, the device 2 includes a detector 8 that detects peak optical absorption information of a compound 4. In this particular embodiment, the depth of penetration of the compound 4 is known. For example, the compound 4 may be delivered using a device or system that deposits compounds 4 at a known or pre-set depth level. For removal of the compound 4, the detector 8 need only detect peak optical absorption information of the compound 4. It should be noted, however, that depth detection may be integrated into the detector 8. For example, in the context of tattoo pigment compounds 4, these compounds 4 may migrate within the skin tissue 6 such that the pigment compounds 4 are not concentrated at a single depth. Thus, it may be advantageous to combine the ability to detect peak optical absorption information and depth of penetration into a single detector 8.

Turning back to FIG. 1, the device 2 includes a laser source 12 for delivering radiation to the tissue 6 for the removal (e.g., photofragmentation) of the compound 4. The laser source 12 may include a laser device capable of lasing at desired wavelength(s). The laser source 12 may emit radiation at a fixed wavelength or at a tunable wavelength. Moreover, the laser source 12 may include a single source (e.g., a tunable source) or a plurality of sources (e.g., multiple fixed wavelength sources) in which the wavelength is selected. Preferably, the laser source 12 is a tunable laser source 12 which has a fluence level at or above 1 J/cm². In addition, assuming that the beam of radiation from the laser source 12 is focused to about 10 μm in radius, the pulse energy of the radiation should be on the order of 1 μJ. In addition, the tunable laser source 12 is preferably tunable between the range of about 500 nm to about 650 nm. The laser source 12 is preferably coupled to a delivery member 20 which is used to direct the radiation into the tissue 6. The delivery member 20 may include, for example, one or more optical pathways such as an optical fiber or a bundle of fibers (e.g., multimode fiber). One skilled in the art will appreciate that the particular delivery member 20 used may include any of those commonly used to transport laser radiation to a target location that is located remote from the laser source 12. Light exits the delivery member 20 where it passes through the tissue 6 to a depth (d) where the compound of interest (e.g., tattoo pigment compound 4) is located. When the laser radiation contacts the compound 4, the compound 4 is degraded into photofragments. Where the compound 4 is a tattoo pigment compound 4, the particles making up the tattoo pigment compound 4 are fragmented into photofragments, thereby degrading and removing the color associated with the pigment compound 4.

Still referring to FIG. 1, a controller 22 is preferably used to control both the detector 8 and laser source 12. In addition, the controller 22 is used to acquire and process data collected in the detector 8 portion of the device 2. In one aspect of the invention, the controller 22 acquires depth (d) and/or peak optical absorption data and based on this data tunes or selects the laser source 12 to the appropriate wavelength. The controller 22 preferably operates on a real-time (or near real-time) basis, thus allowing the device 2 to monitor any absorption peak changes and depth variations using the real-time detection scheme and consequently, adjust laser parameters automatically on a real-time basis. The controller 22 is preferably microprocessor-based and may comprise, for example, a personal computer or the like (not shown).

In many instances, a tattoo may be formed from a plurality of different tattoo pigment compounds 4. For example, an orange colored tattoo may include red and yellow pigment compounds 4. In one aspect of the invention, the laser source 12 may be tuned to remove a first tattoo pigment compound 4 (e.g., red). After the first tattoo pigment compound 4 has been removed or reduced below an acceptable threshold level, the laser source may be tuned to remove the second tattoo pigment compound 4 (e.g., yellow). In this regard, the various constituent pigment compounds 4 may be removed on a sequential basis. In an alternative embodiment, the different pigment compounds 4 may be removed simultaneously. For example, a first laser source 12 may be used to remove a first pigment compound 4 while a second laser source 12 may be used to remove a different pigment compound. The process may take place simultaneously or near-simultaneously. For example, in the case of a pulsed laser source 12, the pulsed laser radiation may alternate between the different laser sources operating at different wavelengths.

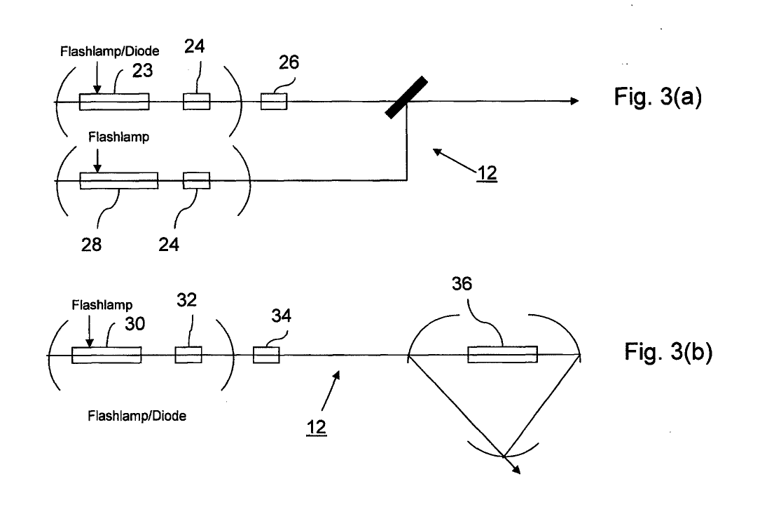

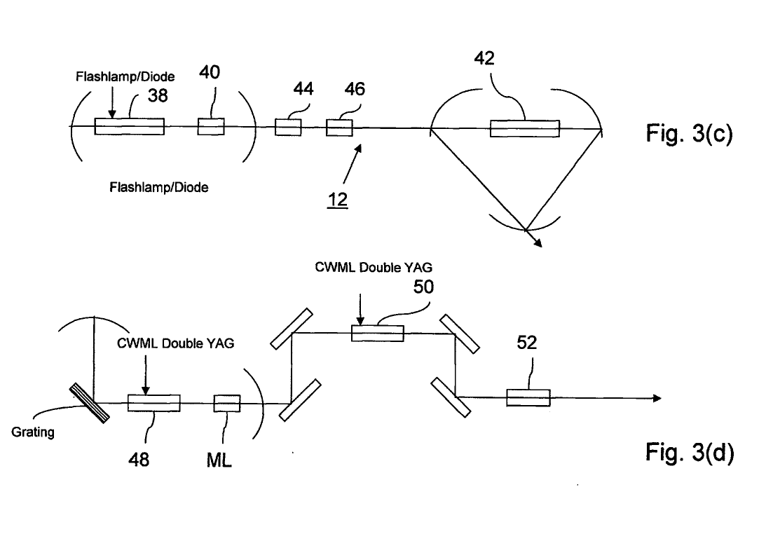

FIG. 3(a) illustrates one exemplary laser source 12 according to one embodiment of the invention. It should be understood, however, that other laser sources 12 different from the specific embodiments illustrated herein may be used in accordance with the methods and systems disclosed herein. According to this embodiment, the laser source 12 includes two lasers, namely, a double Nd:YAG laser (operating at 532 nm) and a ruby laser (operating at 694 nm). The advantages of this particular embodiment is the ease of implementation because of the commercial availability of the system components. In addition, the dual wavelengths would allow a better performance than a single wavelength system. As seen in FIG. 3(a), a flashlamp/diode pumped Nd:YAG laser 23 is operating under pulse mode via a Q-switch 24. This particular laser 23 emits radiation at 1064 nm with 10 ns and 30 mJ pulses. The light frequency is doubled to 532 nm by the KTP (potassium titanium phosphate) nonlinear crystal 26 with about 50% efficiency. The resulting beam has a power of about 15 mJ which is ample for 1 μJ applications. Still referring to FIG. 3(a), the laser source 12 includes a flashlamp-pumped ruby laser 28 under operation of Q-switch 24 which operates with 2 ns and 160 mJ pulses at 694.2 nm.

FIG. 3(b) illustrates a laser source 12 according to another preferred aspect of the invention. The laser source 12 includes a tunable OPO (optical parametric oscillator) ruby laser 30 operating in pulsed mode via a Q-switch 32. The laser 30 emits radiation at 694.3 nm in 2 ns pulses at 160 mJ. The light frequency may be doubled by a nonlinear BBO (beta-BaB₂O₄) crystal to 347 nm with about 50% efficiency. The resultant radiation beam is used to pump a nonlinear OPO of BBO 36. Generally, one photon of 347 nm is split into two, each of lower energy. By dividing energy differently between the daughter photons, tunability can be achieved over a range of around 460 nm to 600 nm. Assuming an efficiency of around 40%, it is possible to obtain a 2 ns pulse having a power of 32 mJ.

FIG. 3(c) illustrates a laser source 12 according to yet another embodiment. The laser source 12 includes an OPO tunable Nd:YAG laser 38 that is operating in pulsed mode via a Q-switch 40. Tunability is achieved by using a nonlinear OPO 42 formed from BBO. A 30 mJ, 10 ns pulse at 1064 nm can be quadrupled by nonlinear KTP and BBO crystals 44, 46, respectively, to 266 nm with about 25% efficiency. The 266 nm pulses are then is used to pump a nonlinear OPO 42 of BBO. One photon of 266 nm is split into two, each of lower energy. By dividing energy differently between daughter photons, tunability can be achieved over a range of about 460 nm to 600 nm. Assuming an efficiency of around 40%, it is possible to obtain a 2 ns pulse having a power of 3 mJ.

FIG. 3(d) illustrates a laser source 12 according to still another aspect of the invention. The laser source 12 includes a continuous wave mode-locked Ti:Sapphire laser 48 (using modelocker ML). Tunability is achieved by the gain medium which may include, for example, Cr:Forsterit or BaSO₄:Mn. The output of the continuous wave mode-locked Ti:Sapphire laser 48, which may include a 50 fs pulse of 100 μJ light, is amplified. The amplification is done by the pulse-stretching-compression technique such as that disclosed in Chen et al., Chirped Amplification of 50 fs 100 μJ Pulse at the Repetition Rate of 5 kHz, Proc. SPIE, Vol. 2869, pp. 508-514, May, 1997, which is incorporated by reference as if set forth fully herein. The stretched pulse is amplified by another Ti:Sapphire laser 50 followed by compression. The amplified pulse is sent to a nonlinear crystal BBO 52 for frequency doubling. Because the Ti:Sapphire laser 48, 50 can be tuned within the 930 nm to 1200 nm range, a pulse of 50 μJ within the range of about 460 nm to about 600 nm is achievable.

FIG. 1 illustrates an integrated system or device 2 according to one embodiment. Skin tissue 6 is disclosed containing one or more compounds 4 in the form of tattoo pigment compounds 4. The detector 8 may include a spectral optical coherence tomography (OCT) system of the type disclosed in FIG. 2. The system 2 is able to image the tattoo pigment distribution inside the skin tissue 6 with high resolution (˜1 μm). The detector 8 coupled to the controller 22 permits real-time or near real-time access to spatial and spectral information on the tattoo pigment compounds 4. For example, the detector 8 may be able to determine the depth (d) of the tattoo pigment compounds 4 within the skin tissue 6 as well as determine the absorption peak(s) of the compounds 4 contained therein. In a preferred embodiment, the laser source 12 (e.g., Ti:Sapphire laser source) may be combined or even integrated with the detector 8. In particular, the laser source 12 may be operated at low power and an ultra-short pulse of light is sent to the OCT detector 8. After computer analysis, for example, using controller 22, the system can tune to the optical wavelength and focus to the pigment for high-power ablation and/or photofragmentation. The system 2 may operate using a number of cycles which may include detection followed by one or more lasing operations. The area of interest may be subject to additional detection operations to detect, for example, remaining compounds 4 or photofragments of compounds 4. This can then be followed by additional imaging/lasing cycles to analyze the ablation/photofragmentation performance.

It should be understood that the system or device 2 may include one laser source 12 for multiple compounds 4, or alternatively, the device 2 may include multiple laser sources 12 for a single compound 4. The device 2 may incorporate well known switching mechanisms to incorporate multiple laser sources 12.

In one embodiment, the device 2 can be used to reduce or increase the concentration of one or more pharmaceutical compounds 4 within tissue 6 such as skin tissue 6. In one aspect, a pharmaceutical compound 4 (or multiple compounds 4) is deposited or otherwise administered locally within the skin tissue 6. A laser source 12 is used to illuminate the region of skin 6 containing the pharmaceutical compound 4. The laser radiation interacts with and breaks down the pharmaceutical compound 4, thereby decreasing (or removing entirely) the localized concentration of the pharmaceutical compound 4 in the skin tissue 6. The device 2 may have a plurality of detection/lasing cycles to reduce the concentration of the pharmaceutical compound 4 below a pre-set threshold value.

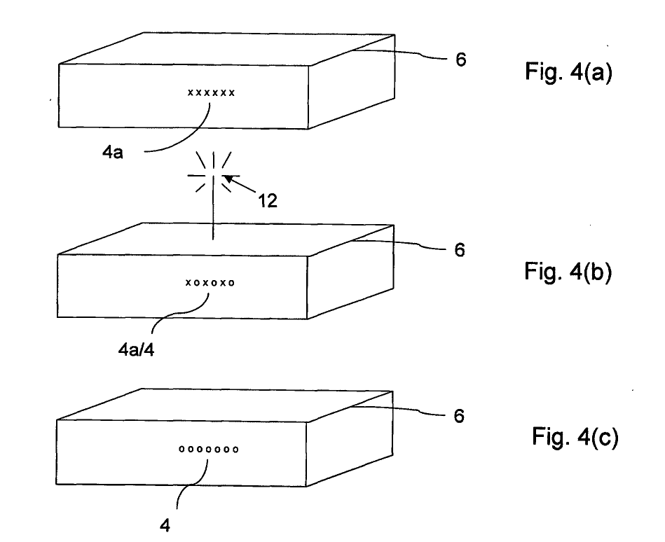

In another aspect, the device 2 is used to deliver or transform one or more pharmaceutical compounds 4 in tissue 6 such as skin tissue. In this embodiment, one or more pharmaceutical precursor compounds 4a, such as that shown in FIG. 4(a), are delivered to a subject such as a patient. The pharmaceutical precursor compound 4a may be delivered or administered locally, e.g., directly in the skin tissue 6 or, alternatively, may be delivered systemically, e.g., via the blood stream or by oral administration. A laser source 12 is then used to illuminate a region of tissue such as skin tissue 6 containing the one or more pharmaceutical precursor compounds 4a. The laser radiation interacts and transforms the pharmaceutical precursor compound(s) 4a into a compound 4 (or multiple compounds) having therapeutic properties. These may include, for example, photofragments. In this regard, radiation is used to initiate or otherwise trigger or modulate the release of a therapeutic pharmaceutical compound 4 located within tissue 6. In one illustrative example, a pharmaceutical precursor compound 4a may be delivered to a subject (e.g., orally or locally to a subject). A selected area of tissue 6, such as, for example, diseased tissue 6 (for example, cancerous tissue) may then be irradiated with laser radiation from the device 2. The laser radiation initiates the transformation of the pharmaceutical precursor compound 4a into a therapeutic pharmaceutical compound 4. The device 2 may cycle through a number of detection/lasing cycles to monitor the concentration of the pharmaceutical precursor compound 4a and/or therapeutic pharmaceutical compound 4.

FIGS. 4(a), 4(b), and 4(c) illustrates the transformation of a pharmaceutical precursor compound 4a into a therapeutic pharmaceutical compound 4. As seen in FIG. 4(a), a portion of tissue 6 contains one or more pharmaceutical precursor compounds 4a (one such compound is shown in FIG. 4(a)). The tissue 6 may include skin tissue 6 although other tissue types are envisioned to fall within the scope of the broad concepts disclosed herein. The region of tissue 6 containing the pharmaceutical precursor compound 4a is irradiated with the laser source 12 as is shown in FIG. 4(b). The laser radiation transform the pharmaceutical precursor compound 4a into a therapeutic pharmaceutical compound 4. Preferably, the region may be monitored using the detector 8 to monitor and/or evaluate the transformation of the pharmaceutical precursor compound 4a. The detector 8 may determine the rate of formation/depletion of the compounds 4, 4a and/or their absolute concentrations within the tissue 6.

In still another aspect of the invention, laser radiation from laser source 12 may be used to release one or more pharmaceutical compounds 4 (or precursor compounds 4a) contained inside cellular structures located in tissue (e.g. cells). The laser radiation may be used to lyse or otherwise cause the cells or other structures to release the one or more pharmaceutical compounds 4 (or precursor compounds 4a). The one or more pharmaceutical compounds 4 or precursor compounds 4a can then be used for localized or even systemic therapeutic applications.

With respect to use of the device 2 for the administration and removal of tattoos, it is preferable that tattoo administration should be performed using pigments 4 that are safe and completely (or nearly completely) removable. Preferably, a motorized or other automated tattooing instrument (not shown) may be used to implant the tattoo pigment compounds 4 at known depth (d) in the skin 6 which is pre-determined to allow for both permanence and ease of removal. In this regard, an integrated system may be provided that permits the tattooing and removal with a single device. One aspect of the device would be used for depositing the tattoo pigment compounds 4 while another aspect is used for the removal of the tattoo pigment compounds 4. For the removal of tattoos, the detector 8 is used to determine the depth (d) and/or absorption peak of the pigment 4. Based on these parameters, the laser source 12 is tuned as appropriate and aimed at the tattoo pigment compound 4. The laser source 12 is preferably optimized in wavelength and fluence level for the photofragmentation process. For example, in one aspect of the device 2, the detector 8 monitors in real-time or near real-time the changes in the optical properties of the tattoo pigment compound 4 and adjusts the wavelength of the laser source 12 to achieve maximum energy transfer to the tattoo pigment compound 4 (or photofragments of the compound) while at the same time minimizing energy transfer into the surrounding tissue 6.

While embodiments of the present invention have been shown and described, various modifications may be made without departing from the scope of the present invention. The invention, therefore, should not be limited, except to the following claims, and their equivalents.

What is claimed is:

1. A device for removing a compound in tissue comprising:
a detector for detecting at least one optical property of the compound in the tissue;
a laser source, wherein the wavelength of the laser source is chosen based on the at least one optical property of the compound in the tissue; and
a delivery member for delivering radiation from the laser source to the compound in the tissue.

2. The device according to claim 1, wherein the detector detects the depth of the compound within the tissue.

3. The device according to claim 1, wherein the at least one optical property comprises peak optical absorption information.

4. The device according to claim 1, wherein the at least one optical property comprises fluorescence peak information.

5. The device according to claim 1, wherein the laser source is tunable.

6. The device according to claim 5, wherein the laser source is tunable over a series of wavelength ranges having a bandwidth of approximately 150 nm.

7. The device of claim 1, wherein the compound comprises a pharmaceutical compound.

8. The device of claim 1, wherein the compound comprises a pharmaceutical precursor compound.

9. The device of claim 1, wherein the compound comprises a tattoo pigment compound.

10. The device according to claim 9, wherein the tattoo pigment compound comprises Chicago Sky Blue 6B.

11. The device according to claim 9, wherein the tattoo pigment compound comprises Methyl Red.

12. The device according to claim 9, wherein the tattoo pigment compound comprises Phenolphthalein.

13. The device according to claim 9, wherein the tattoo pigment compound comprises Janus Green B.

14. The device according to claim 9, wherein the tattoo pigment compound comprises Crystal Violet.

15. The device according to claim 9, wherein the tattoo pigment compound comprises Cresyl Violet Perchlorate.

16. The device according to claim 9, wherein the tattoo pigment compound comprises Chrysophenine.

17. The device according to claim 9, wherein the tattoo pigment compound comprises Fast Black K Salt (Azoic Diazo No. 38).

18. The device according to claim 1, wherein the laser source comprises a tunable Nd:YAG laser.

19. The device according to claim 1, wherein the laser source comprises a tunable Ti:Sapphire laser.

20. The device according to claim 1, wherein the laser source has a fluence level at or above 1 J/Cm².

21. The device according to claim 5, wherein the tunable laser source is continuously tunable over the wavelength range of about 500 nm to about 650 nm.

22. The device according to claim 5, further comprising a controller for controlling the detector and tunable laser source.

23. The device according to claim 1, wherein the detector detects the depth and peak optical absorption of the compound in the tissue.

24. The device according to claim 23, wherein the wavelength of the tunable laser source is tuned based on the peak optical absorption of photofragments of the compound in the tissue.

25. The device according to claim 1, wherein the detector comprises a spectral optical coherence tomography system.

26. The device according to claim 1, wherein the laser source comprises a plurality of laser sources operating at a fixed wavelength.

27. A method of administering a tattoo, the method comprising inserting a pigment into the dermis layer of skin at a pre-determined depth level, the pigment being selected from the group consisting of Chicago Sky Blue 6B, Methyl Red, Phenolphthalein, Janus Green B, Crystal Violet, Cresyl Violet Perchlorate, Chrysophenine, and Fast Black K Salt (Azoic Diazo No. 38).

28. A method of removing tattoo pigment in tissue, the method comprising the steps of:
providing a detector;
providing a tunable laser source coupled to a delivery member for delivering radiation from the tunable laser source to the tattoo pigment in the tissue;
detecting the peak optical absorption of the tattoo pigment in the tissue with the detector;
adjusting the wavelength of the tunable laser source based on the peak optical absorption of the tattoo pigment in the tissue; and
delivering radiation at an adjusted wavelength from the tunable laser source to the tattoo pigment in the tissue with the delivery member.

29. The method of claim 28, further comprising the steps of:
detecting the peak optical absorption of photofragments of the tattoo pigment in the tissue with the detector;
adjusting the wavelength of the tunable laser source based on the peak optical absorption of the photofragments of the tattoo pigment in the tissue; and
delivering radiation at an adjusted wavelength from the tunable laser source to the photofragments of the tattoo pigment in the tissue with the delivery member.
